(19) United States
(12) Patent Application Publication
(10) Pub. No.: US 2004/0127931 A1
(43) Pub. Date: Jul. 1, 2004

(54) Cervical dilator

(75) Inventors: Geoffrey C. Kincaid, Rockford, TN (US); Matthew R. Otten, Cincinnati, OH (US)

Correspondence Address: WOOD, HERRON & EVANS, LLP, 2700 CAREW TOWER, 441 VINE STREET, CINCINNATI, OH, 45202, US

(73) Assignee: Geoffrey C. Kincaid, Rockford, TN

(21) Appl. No.: 10/216,114

(22) Filed: Aug. 9, 2002

(51) Int. Cl. A61M 29/00

(52) U.S. Cl. 606/193; 606/198

(57) ABSTRACT

A Cervical Dilator which performs dilatation of the uterine cervix through the use of an inflatable balloon powered by compressed gas. The balloon is manufactured of non-compliant materials, so that its dimensions when fully inflated are predetermined. Physiologic saline or other liquid is introduced into the ballon by a piston or diaphragm that is driven by a self-contained compressed gas source. This arrangement places a barrier between the patient and the gas source, thereby eliminating the possibility ot gas embolization. The rate at which compressed gas is delivered to the system is precisely controlled by a trigger and valve mechanism. This allows the physician to precisely, and progressively, inflate the ballon and consequently dilate the cervix to the chosen caliber.

### FIELD OF THE INVENTION

This invention relates generally to cervical dilatation, and specifically to a device to achieve such dilatation.

### BACKGROUND OF THE INVENTION

In gynecological and obstetrical practice, it is frequently necessary to dilate the cervical canal for performing many medical and surgical procedures. Cervical dilatation is currently accomplished using tapered rods called dilators. Prior to insertion of the first dilator the cervix must be stabilized. This is usually accomplished using an instrument called a single tooth tenaculum, a scissors like device with two opposing points at its tip. The tenaculum is usually positioned so that, when closed, the pointed tips puncture the cervix on the anterior side, leaving the cervical opening accessible just beneath. The operator also uses the tenaculum to apply counter traction as the dilators are inserted. Successively thicker rods are used until the desired amount of dilatation has been achieved.

As simple as this procedure seems, complications are common. Since the canal cannot be directly visualized, the operator must depend entirely upon his sense of touch in guiding the tip of the dilator through the canal and into the uterine cavity, and again on his sense of touch to stop pushing and withdraw the dilator before passing it through the top of the uterus. In most cases this is not difficult. In the non-pregnant, reproductive age woman the canal is naturally three to four millimeters in diameter, the dilators can be inserted with relative ease, and the uterine fundus is firm enough to be easily felt. In some women, however, particularly post-menopause and those who have had surgery for cervical disease, the cervix may be narrowed and scarred. As greater force is needed to pass the dilators, it becomes much more difficult to know whether you are actually dilating the cervix, or creating a false channel instead. In addition, the application of greater force may cause the tenaculum to tear through the tissue, resulting in laceration of the cervix. Because the procedure is blind, the best way for the operator to know that he has actually dilated the cervix, and not perforated the back wall, is to continue pushing the dilator forward until he feels the resistance when it hits the uterine wall at the top of the cavity. When the cervix is dilated during pregnancy, as for pregnancy termination, the uterine wall is much thinner and softer, and therefore much harder to detect by feel.

The vast majority of uterine perforations cause no significant damage, and heal without any permanent ill affect. In fact, before hysteroscopy, nearly all perforations were merely suspected, rather than known for certain. The operator would suddenly notice no resistance as the tip of the dilator reached the point where it should have been stopped by the uterine wall. In most cases the procedure could be successfully completed anyway, and no one but the operator would have any way to know what had happened.

The ability today to perform complicated intrauterine procedures using the hysteroscope has made perforation both more likely and more dangerous. It is made more likely because the multi-channel operative hysteroscope requires a greater degree of dilatation than was required for simple curettage. At about nine or ten centimeters the cervix has been stretched and thinned to the point that further dilatation can easily lacerate the cervix. Without the ability to apply counter-traction, no further dilating can take place. In addition, since operative hysteroscopy requires the use of fluids under pressure, the procedure must be terminated as soon as a perforation is detected. If not, large volumes of fluids, as well as any tissue surgically removed, will simply be washed directly into the peritoneal cavity.

It is therefore desirable to provide a device utilized as a dilator of the human cervix to reduce the possibility of injury or harm to the patient that may results in complications from the intended procedure.

It is still further desirable to provide a device that is intuitive and easy to use by the medical practitioner.

It is also desirable to have operation of the device in such manner that dilatation of the cervix is precisely controlled.

It is further desirable to reduce the need for considerable skill by the physician for such dilatation and to improve the visibility and placement of the device in the intended environment.

It is therefore desirable to provide such benefits in a design for a device utilized as a dilator of the human cervix that is simple in construction, affordable to manufacture, uses existing technologies, is easily sterilized, produces practical benefits, and is disposable.

### SUMMARY OF THE INVENTION

Although prior art devices and techniques exist, the present invention relates to a novel design, a device utilized as a dilator regarding various body cavities, and in particular, the human uterine cervix. This novel design achieves certain advantages over said prior art devices that are neither taught nor disclosed by said prior art and addresses the above noted desires in the art.

This invention relates to a new design of a device used in the dilatation of the human cervix, or other various body cavities, for medical procedures. Dilatation of the cervix is achieved through the inflation of a balloon that is introduced into the cervix by the device. The balloon is mounted distally to the embodiment of the device and is inflated with saline or other liquid or gas. The distal tip of the device can be curved for assisted access into the cervix. The saline is driven into the balloon by compressed gas that is self-contained within the device or driven by an external source.

Prior to insertion of the dilator the cervix must be stabilized with a tenaculum. The tenaculum is positioned and held secure by a lock and ratchet feature located on the device, leaving the cervical opening accessible just beneath. The balloon is positioned into the cervix at the desired location and inflation begins. As the balloon inflates it presses on the wall of the cervix and expands the cervical opening until complete dilatation is achieved. Once the procedure is complete the balloon is deflated and easily removed from the intended environment.

The accompanying drawings, description and claims will serve to explain the principles of the invention.

### BRIEF DESCRIPTION OF THE DRAWINGS

FIG. 1 is a side perspective view of a device in accordance with the one embodiment of the invention.

FIG. 2 is a side view, in partial section, of the device in FIG. 1.

FIG. 3 is a cross-sectional view of a distal tip and balloon for the devices In FIGS. 1 and 2.

FIG. 4 is a cross-sectional view of a saline chamber of the devices in FIGS. 1 and 2.

FIG. 5 is a cross-sectional view of a deflation chamber of the devices in FIGS. 1 and 2.

### DETAILED DESCRIPTION OF THE EMBODIMENTS

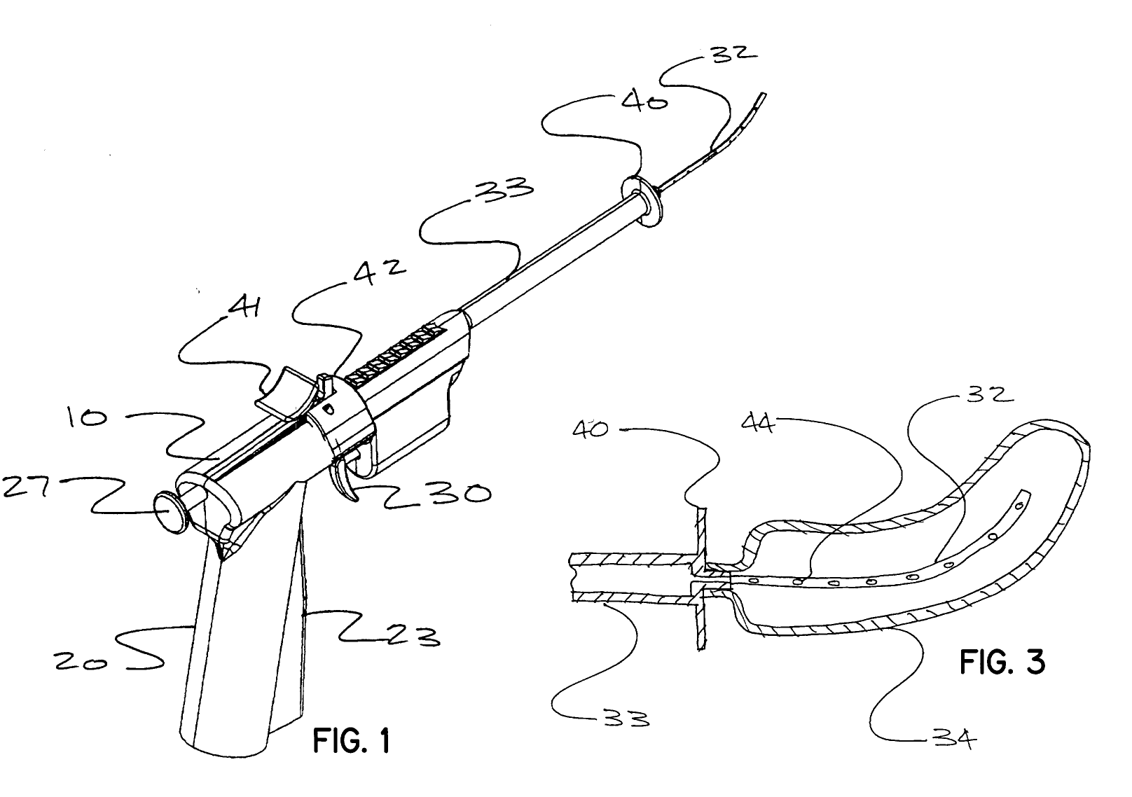

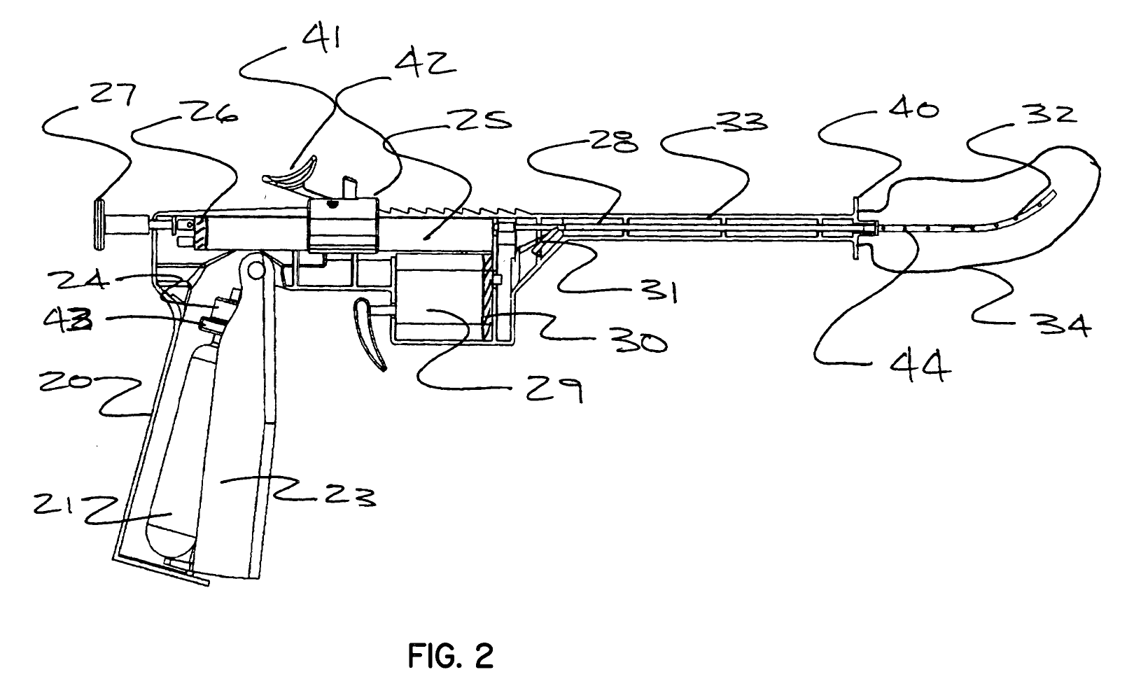

Referring now more particularly to one embodiment of the invention illustrated in FIGS. 1 and 2 of the accompanying drawings. In one embodiment of the invention, a housing 10 is in the general shape of a gun, and has a handle portion 20 and a barrel portion 33. Other shaped housings may also be utilized. In the illustrated embodiment, a lock 41 and ratchet slide structure 42 are utilized to position and secure a tenaculum in place during a procedure utilizing the invention. The lock 41 is configured to engage a tenaculum. The tenaculum might also be manually or otherwise secured.

For dilatation, an inflatable structure, such as a balloon 34 is mounted distally at an end of the barrel portion 33 of the device. Specifically, the device includes a curved device tip 32 which is coupled to tubular structures 28 and discussed below for delivering an inflation fluid to the inflatable structure or balloon 34. The balloon is operably coupled to the tip 32 and is inflated with saline or other liquid or gas through holes or ports 44 in the curved tip 32 as shown in FIG. 3. One embodiment of the balloon has an elongated cylindrical shape allowing for easy introduction into the cervix and even force distribution during inflation. In one embodiment, due to cervical anatomy, the length of the balloon is around 40 to 50 mm. The balloon may have different inflation diameters, for example ranging between 8 mm and 15 mm. The curved tip 32 of the device may be semi-rigid so that it can be shaped by the operator to conform to the direction of the individual patient's cervical canal. The balloon is secured lo to the housing end around the tip 32 with a suitable method, such as an adhesive.

Referring again to FIG. 2, a compressed gas storage chamber 21 is positioned in the handle portion 20 of the housing. In one embodiment, the device may comprise an insertable cartridge system which includes an insertable air cylinder or cartridge, such as a CO2 cartridge that forms the chamber 21. The insertable cartridge design reduces or eliminates any likelihood of leakage of the air from the housing and allows for safe and easy handling and assembly of the device. For example, during shipment and storage the cartridge 21 is fully intact and may be separate from the device. The device is then armed or made ready prior to use by positioning and advancing the cartridge 21 so that it is punctured against a needle 43. Another embodiment includes a chamber 21 which is filled by a secondary gas or fluid source and is part of the housing 10.

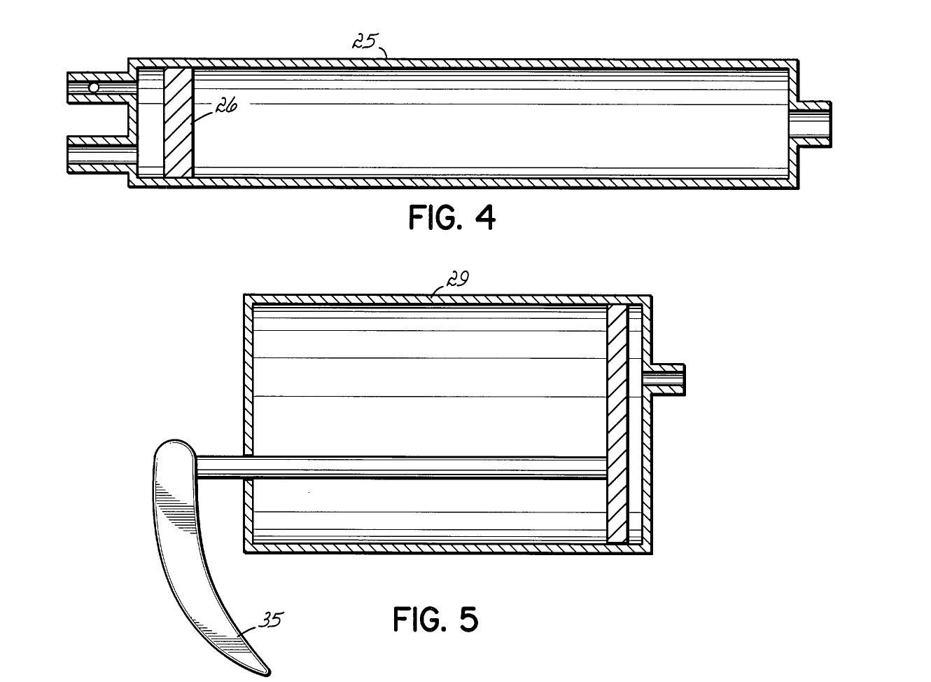

Once the balloon 34 is introduced into the cervix and positioned to the desired depth with the aid of a locator stop 40 on the housing, the physician depresses a trigger 23 on the handle portion to inflate the balloon 34. In one embodiment, the trigger 23 is operably coupled to activate a mechanism 24 that advances a cartridge 21 so that it is punctured against needle 24 and releases compressed gas from the cartridge 21 located in the handle body. Alternatively, the mechanism 24 may include a valve which, when activated, releases air from gas storage chambe 21. The actuation trigger is spring return assisted. As gas is released from the gas storage chamber or cartridge 21, it travels through the mechanism 24 and to a fluid storage chamber 25 to push a piston or diaphragm 26 mounted to slide in the fluid storage chamber 25 as shown in FIG. 4. The piston is positioned or coupled effectively between the gas from the gas chamber and fluid in the fluid chamber. The piston 26, driven by the compressed gas, moves or pushes the inflation fluid, such as saline, into tube 28 and into the tip 32. The fluid exits the tip into the balloon to inflate the balloon and dilate the cervix. For such fluid transfer, the chamber 21 and mechanism 24 are fluidly coupled with the fluid storage chamber 25 such as by one or more tubes (not shown). The fluid storage chamber 25 might also be a cartridge system with physiologic saline in a predetermined volume or cartridge amount to fully inflate the balloon. Since saline is a non-compressible fluid, generally no compression occurs as the piston or diaphragm 26 is driven forward by the compressed gas. The saline is simply transferred from the chamber 25 into the balloon 34 by the movement of the piston or diaphragm under the force of compressed air or gas from chamber 21. As noted, the saline travels from the saline chamber 25 into the balloon 34 through tubes or tubular structures 28.

After the balloon and cervix have been fully dilated, deflation of the balloon may be achieved. In one embodiment, deflation is done through the use of a manually activated deflation cylinder 29. Manual activation is achieved through the use of a plunger or piston 30 located in the housing of the deflation cylinder 29. The plunger is moved or activated by a trigger or handle 35 coupled with the plunger as shown in FIG. 5. By manually manipulating the handle 35, the plunger 30 is retracted and the saline in the balloon and tube 28 is then drawn out by vacuum through tubing 28 and 31 and into the deflation cylinder 29. The deflation cylinder can also be used at any time if for any reason it becomes necessary to abort the procedure. Once the balloon is fully deflated and returns to its original shape it can then be readily removed from the cervix and patient.

The pressurized gas may be released from the system using a spring assisted gas release valve 27 and the instrument may be disposed of. The release valve 27 is operably coupled with the chamber 25 to release air or gas therefrom. Specifically, the valve 27 is coupled to the chamber 25 on the side of the piston opposite the fluid, or rather is coupled generally intermediate the piston and the gas chamber 21 to provide a release opening for the compressed gas which drives the piston. Release of the gas through valve 27 allows the piston to retract.

In another embodiment, the fluid is released without the use of a deflation cylinder 29. That is, the embodiment does not utilize active withdrawal of the fluid through a deflation chamber. Deflation of the balloon is achieved through the expiration of the compressed gas in the system. Gas is released from the system using the spring assisted gas release valve 27. Since the internal pressure residing in the balloon and inflation system is greater than the surrounding atmospheric pressure, once the gas is exasperated the saline or other fluid pushes against the piston 26 and returns to the original chamber 25. Once the balloon is fully deflated and returns to its original shape it can then be readily removed from the patient. Any remaining gas is released from the system using the spring assisted gas release valve 27 and the instrument may be disposed of.

While the present invention has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the invention in its broader aspects is not limited to the specific details representative apparatus and method, and illustrative examples shown and described. Accordingly, departures may be made from such details without departure from the spirit or scope of applicant's general inventive concept.

We claim:

1. A device for dilatation of the human cervix and/or other body cavities, comprising:
a housing;
a fluid chamber configured for containing an inflation fluid;
an inflatable structure in fluid communication with the fluid chamber and configured for receiving inflation fluid to be inflated;
a gas chamber configured for containing compressed gas;
a piston slidably mounted and operably coupled between gas from the gas chamber and fluid in the fluid chamber so that release of compressed gas from the gas chamber drives the piston to move inflation fluid into the inflatable structure;
thereby inflating the inflatable structure to dilate a body cavity.

2. The device of claim 1 wherein said piston is mounted in the fluid chamber to slide therein.

3. The device of claim 1 further comprising a tubular structure coupled between the inflatable structure and the fluid chamber for directing inflation fluid to the inflatable structure.

4. The device of claim 1 further comprising a lock for positioning ancillary devices.

5. The device of claim 2 further comprising a tip coupled to the tubular structure and having holes for introducing inflation fluid to the inflatable structure.

6. The device of claim 5 wherein the tip is shapeable.

7. The device of claim 1 further comprising a deflation cylinder coupled to the inflatable structure, the deflation chamber operable for selectively drawing inflation fluid from the inflatable structure to deflate the structure.

8. The device of claim 7 wherein the deflation chamber includes a movable plunger for drawing the inflation fluid.

9. The device of claim 1 wherein the gas chamber includes a compressed gas cylinder.

10. The device of claim 1 further comprising a trigger coupled to the gas chamber for selectively releasing gas from the gas chamber.

11. The device of claim 1 further comprising a stop structure coupled to the housing and positioned proximate the inflatable structure for controlling the positioning of the inflatable structure in a body cavity.

12. The device of claim 1 further comprising a gas release valve coupled to release the compressed gas driving the piston.

13. A method for dilating a body cavity comprising:
positioning an inflatable structure within the body cavity with a fluid chamber containing an inflation fluid being in fluid communication with the inflatable structure;
selectively releasing compressed gas from a gas chamber;
with the released compressed gas, driving a piston which is slidably coupled between the gas and the inflation fluid, to thereby move inflation fluid into the inflatable structure to inflate it and dilate the body cavity.

14. The method of claim 13 further comprising directing inflation fluid into the inflatable structure through a tip coupled with the fluid chamber.

15. The method of claim 14 wherein the tip is shapeable, and further comprising shaping the tip.

16. The method of claim 13 further comprising deflating the inflatable structure by drawing inflation fluid from the inflatable structure.

17. The method of claim 16 further comprising drawing the inflation fluid with a deflation cylinder including a retractable plunger.

18. The method of claim 13 further comprising releasing, with a gas release valve, the gas which drives the piston so the inflatable structure may deflate.
